(19) United States
(12) Patent Application Publication
(10) Pub. No.: US 2023/0365477 A1
(43) Pub. Date: Nov. 16, 2023

(54) Catalytic Desulfurization of Marine Gas Oil and Marine Diesel Oil under Methane Environment

(71) Applicant: Kara Technologies Inc., Calgary AB (CA)

(72) Inventors: Hua Song, Calgary (CA); Hao Xu, Calgary (CA); Zhaofei Li, Calgary (CA); Yimeng Li, Calgary (CA)

(21) Appl. No.: 18/355,466

(22) Filed: Jul. 20, 2023

Related U.S. Application Data
(63) Continuation of application No. PCT/IB2022/050716, filed on Jan. 27, 2022.
(60) Provisional application No. 63/143,279, filed on Jan. 29, 2021.

(51) Int. Cl. C07C 2/84

(52) U.S. Cl. CPC C07C 2/84; C07C 2529/48

(57) ABSTRACT

A method of desulfurizing a sulfur-containing hydrocarbon feedstock includes introducing the sulfur-containing hydrocarbon feedstock within a reactor in the presence of a gas atmosphere and a catalyst structure, where the catalyst structure comprises a zeolite porous support structure including gallium (Ga) and molybdenum (Mo) loaded in the zeolite porous support structure. The gas atmosphere can include methane. At least 50% of sulfur content can be removed from the feedstock as a result of the desulfurizing method.

### CROSS-REFERENCE TO RELATED APPLICATIONS

This application is a continuation of International Patent Application Number PCT/IB2022/050716, filed Jan. 27, 2022, which claims priority to U.S. Provisional Patent Application Ser. No. 63/143,279, filed Jan. 29, 2021, the disclosures of which are incorporated herein by reference in their entireties.

### FIELD

The present invention is directed toward the desulfurization of oils and, in particular, desulfurization of marine gas oils and marine diesel oils, to control fuel emissions during the use of such oils.

### BACKGROUND

Recent regulations set forth by the International Maritime Organization (IMO) require a limit of sulfur content in bunker fuels used on ships within designated emission control areas (ECAs) at <0.1% m/m (mass by mass), while the limit for the sulfur content outside ECAs is tightened to 0.50% m/m. The new regulation will directly benefit the environmental protection and human health, reducing sulfur oxide (SOₓ) emissions globally due to maritime activities. However, this will also significantly impact the marine fuel industry due to the predictable highly increased fuel refining and upgrading cost.

To meet the 0.50% m/m sulfur content limit, high-sulfur fuel oil (HSFO) such as intermediate fuel oil (IFO) and heavy fuel oil (HFO) (which was the most commonly used marine fuel) may continue to be used only if an exhaust gas cleaning system (EGCS) is installed on vessels. Otherwise, the fuel has to be switched to use very-low-sulfur fuel oil (VLSFO) such as marine diesel oil (MDO) with sulfur content below 0.5%. Furthermore, an even stricter sulfur limit<0.10% m/m is applied inside ECAs, where the ship engines have to consume ultra-low-sulfur fuel oil (ULSFO) where low-sulfur marine gas oil (LSMGO) is the only feasible option at the current stage.

Based upon the projection model shown in the assessment of fuel oil availability report published by the IMO, the estimated annual bunker fuel demands of ULSFO, VLSFO and HSFO are 33 to 48 million tons, 198 to 200 million tons, and 14 to 38 million tons, respectively. With the new regulations in place, VLSFO and ULSFO will likely be used to meet the major demand among the bunker fuel industry and occupy most of the market share. The price of marine fuel is greatly affected and determined by its sulfur content, making it economically promising to upgrade bunker fuels to achieve a reduced sulfur content.

Hydrodesulfurization is commonly practiced in the industry to break down the C—S bond under a hydrogen environment and convert sulfur-containing species in the bunker fuel in the form of H₂S. However, this process must consume hydrogen, which is not naturally available. Most of the hydrogen utilized in the industry is obtained through steam reforming of natural gas at high operating temperatures (e.g., greater than 800° C.) and high operating pressures (e.g., 1.5-3.0 MPa). The hydrodesulfurization process is also executed at high pressure (e.g., as high as 13 MPa or greater), resulting in increased operating costs.

Instead of using hydrogen, if natural gas can be directly used as the hydrogen source, the steam reforming process can be skipped, leading to a significant cost reduction of the desulfurization process. The utilization of methane, the principal component of natural gas, as an alternative hydrogen source can be applied as a methanotreating process of the oil to produce extra products with reduced CO₂ emissions, thereby making the upgrading under methane even more economically favorable and environmentally friendly. However, it is more challenging to activate methane instead of hydrogen for upgrading and desulfurization of oil production due to methane having a stable structure and being more inert (with a strong C—H bond).

Because of the foregoing, it would be advantageous to provide a process for desulfurization of an oil product, such as a marine gas oil (MGO) or a marine diesel oil (MDO) utilizing methane which is more energy-efficient, cost-effective, and environmentally friendly concerning emissions associated with the methanotreating process for the oil product.

### BRIEF SUMMARY

In accordance with embodiments described herein, a method of desulfurization of a sulfur-containing hydrocarbon feedstock is described herein. The method comprises introducing the sulfur-containing hydrocarbon feedstock within a reactor in the presence of a gas atmosphere and a catalyst structure, where the catalyst structure comprises a zeolite porous support structure including gallium (Ga) and molybdenum (Mo) loaded in the zeolite porous support structure. The gas atmosphere can comprise methane. At least 50% of sulfur content can be removed from the feedstock as a result of the desulfurizing method.

The above and still further features and advantages of the present invention will become apparent upon consideration of the following detailed description of specific embodiments thereof.

### BRIEF DESCRIPTION OF THE DRAWINGS

FIG. 1 depicts a plot of simulated distillation analysis curves for marine gas oil (MGO) and its products after being subjected to desulfurization/upgrading treatment according to processes as described herein.

FIG. 2 depicts a plot of simulated distillation analysis curves for marine diesel oil (MDO) and its products after being subjected to desulfurization/upgrading treatment according to processes as described herein.

### DETAILED DESCRIPTION

In the following detailed description, while aspects of the disclosure are disclosed, alternate embodiments of the present disclosure and their equivalents may be devised without parting from the spirit or scope of the present disclosure. It should be noted that any discussion herein regarding “one embodiment”, “an embodiment”, “an exemplary embodiment”, and the like indicate that the embodiment described may include a particular feature, structure, or characteristic and that such particular feature, structure, or characteristic may not necessarily be included in every embodiment. In addition, references to the foregoing do not necessarily comprise a reference to the same embodiment. Finally, irrespective of whether it is explicitly described, one of ordinary skill in the art would readily appreciate that each of the particular features, structures, or characteristics of the given embodiments may be utilized in connection or combination with those of any other embodiment discussed herein.

Various operations may be described as multiple discrete actions or operations in turn, in a manner that is most helpful in understanding the claimed subject matter. However, the order of description should not be construed to imply that these operations are necessarily order-dependent. In particular, these operations may not be performed in the order of presentation. Operations described may be performed in a different order than the described embodiment. Various additional operations may be performed and/or described operations may be omitted in additional embodiments.

For the present disclosure, the phrase “A and/or B” means (A), (B), or (A and B). For the present disclosure, the phrase “A, B, and/or C” means (A), (B), (C), (A and B), (A and C), (B and C), or (A, B and C).

The terms “comprising,” “including,” “having,” and the like, as used with respect to embodiments of the present disclosure, are synonymous.

The importance of providing a low sulfur fuel oil and, in particular, fuel oils used in the marine industry such as marine diesel oil (MDO) and marine gas oil (MGO), has been previously noted herein. In accordance with example embodiments described herein, processes are described for effective desulfurization of fuel oil in the presence of methane and utilizing a suitable catalyst to yield an upgraded, low sulfur fuel oil product. As described herein, a catalyst structure is provided in the desulfurization process that comprises a porous aluminosilicate material loaded with a combination of metals that includes gallium (Ga) and molybdenum (Mo). The combination of the specified catalyst including Ga—Mo enhances the activation of methane, aromatization of the feedstock oil, and conversion of sulfur-containing groups, particularly when MDO or MGO with a higher sulfur content is charged as the feed. Further, utilizing a porous aluminosilicate support structure, such as a ZSM-5 structure having uniform cylindrical morphology (UZSM-5), prevents or inhibits over-cracking of molecules in the oil feedstock.

The process described herein results in desulfurization of the fuel oil feedstock resulting in a conversion of at least about 50% by weight of the sulfur content in the feedstock (i.e., a 50% reduction by weight of an amount of sulfur in the feedstock), which is much greater than that which is achieved using conventional desulfurization processes (e.g., in the presence of hydrogen or nitrogen). Further, the use of methane in the process not only improves the desulfurization performance but also suppresses coking and over-cracking of the feedstock as well as increasing the liquid product yield (which is likely due to methane incorporation in the product molecules).

Any fuel oil or other sulfur-containing hydrocarbon feedstock can be upgraded and benefit from the desulfurization process described herein. Some examples of fuel oils include crude oils such as the following:
Marine gas oil (MGO) is a distillate of commercial crude oil for maritime activities produced from refineries with a boiling point range of 350˜500° C. and maximum sulfur content of 1 wt %.
Heavy fuel oil (HFO) is a category of fuel oils of a tar-like consistency, also known as bunker fuel or residual fuel oil (RFO), which is the result of the remnant from the distillation and cracking process of petroleum. It is contaminated with several different compounds including aromatics sulfur and nitrogen, making emission upon combustion more polluting compared to other fuel oils. It is predominantly used as a fuel source for marine vessel propulsion due to its relatively low cost compared to cleaner fuel sources such as distillates. The maximum density can be as high as 1010 kg m⁻³ at 15° C. and the maximum viscosity can be 700 cSt at 50° C. The sulfur content can reach 5 wt %.
Marine diesel oil (MDO) generally describes marine fuels that are composed of MGO and HFO. Marine diesel is similar to diesel fuel but has a higher density, similar viscosity, and the maximum permissible sulfur content of 3.5 wt %.
Light, petroleum ether, petroleum spirit, and petroleum naphtha.
medium crude oil (or medium oil)—crude oil having an API gravity ranging between 22.3° API and 31.1° API. crude oil (or light oil)—crude oil having an API gravity of 31.1° API or higher. The light crude oils generally have a dynamic viscosity of less than 2×10³ cP (mPa·s). Types of light crude oils can be further categorized into very light oils including, without limitation, jet fuel, diesel fuel, gasoline, keroseneMedium crude oils typically have a higher viscosity in relation to light crude oils, the dynamic viscosity is often within the range of 2×10³-2×10⁴ cP (mPa·s).
Heavy crude oil (or heavy oil)—crude oil having an API gravity ranging between 10° API and 22.2° API. Heavy crude oils typically have a higher viscosity in relation to medium crude oils. In particular, heavy crude oil can have a dynamic viscosity of at least about 1×10⁵ cP (mPa·s). Heavy crude oil also includes extra-heavy oil or bitumen. For example, bitumen (which can be obtained, e.g., in Alberta, Canada) often has an average density of 1.0077 g/cm³, API gravity of 8.9° API, and dynamic viscosity of 2×10⁴-2×10⁶ cP (mPa·s) at atmospheric conditions. Other types of heavy oil include bunker fuel and residual oil or resid (i.e., fuel oil remaining after removal of certain distillates, such as gasoline, from petroleum).

Synthetic fuels (e.g., synthetic oils formed using a Fischer-Tropsch process) or bio-oils formed, from biomass via a pyrolysis process, can also be feedstock for the desulfurization process described herein.

Feedstock fuel oils that particularly benefit from the desulfurization process described herein include oils including heavy oils such as bunker fuel oils, and in particular marine diesel oils and marine gas oils, which have the properties (e.g., viscosity, density API gravity) as previously noted herein for heavy oils.

A catalyst structure that has been determined to be very useful in the desulfurization of hydrocarbon products such as oils and, in particular, heavy oils, MDO and MGO in a methane environment comprises a porous aluminosilicate support structure loaded with a plurality of metals that include at least gallium (Ga) and molybdenum (Mo). The choice of the particular metal species provided in a catalyst for the desulfurization process was achieved as a result of careful analysis based upon the specific heavy oil being upgraded as well as how each metal species behave in a catalyst structure for a particular upgrading process. A series of metal-modified ZSM-5 catalysts with controlled acidity and metal loading types have been developed to upgrade different feedstocks under methane, hydrogen, and/or nitrogen environments. It has been determined that different metal species exhibit different adsorption and activation capacity towards different molecules and their functional groups. The careful tuning of the metal species loaded to the zeolite support is required to optimize the upgrading performance in terms of varied feeds. It has been determined unexpectedly that the combination of Ga and Mo in the amounts described herein within a zeolite support structure provides a synergistic effect in reducing sulfur content in heavy oils or other oils such as MGO and MDO.

In addition to providing an effective reduction in sulfur content within the processed oil (e.g., MGO or MDO), other types of upgrading of the processed oil in relation to the feedstock oil can also be achieved. Some examples of upgrading of a hydrocarbon or oil feedstock and, in particular, an oil feedstock such as an MGO feedstock or an MDO feedstock, include, without limitation, change (e.g., decrease) in density, change (e.g., decrease) in viscosity, change (e.g., decrease) in TAN (total acid number), change (e.g., increase) in an amount (e.g., weight percentage) of one or more aromatic hydrocarbons, change (e.g., increase) in the hydrogen to carbon ratio (H/C ratio), and change (e.g., increase) in cetane number.

The porous catalyst support structure can be synthesized by impregnating or doping the support structure with the selected metals (e.g., utilizing a process such as wet impregnation or ion exchange to adsorb metal ions to the porous surfaces of the support material). The porous aluminosilicate support structure can comprise a zeolite material (e.g., an MFI zeolite structure), such as a ZSM-5 type zeolite (e.g., HZSM-5 zeolite, NaZSM-5 zeolite, etc.), A-type zeolite, L-type zeolite, HY type zeolite, and/or any other suitable zeolite structure. The zeolite material forming the porous support structure can include a SiO₂ to Al₂O₃ ratio in the range of 1-280 (i.e., a ratio of SiO₂ to Al₂O₃ that is 1:1 to 280:1), such as a range of 5-28, or a range of 23-280. The zeolite material can further have a BET surface area in the range of 150 m²/g and 550 m²/g.

In example embodiments, the catalyst support structure can be formed to have a uniform cylindrical morphology, such as a uniform ZSM-5 (UZSM-5) morphology. The UZSM-5 catalyst structure has high silica to alumina molar ratio (e.g., about 80:1), a surface area of 300-450 m²/g, a pore volume of 0.20-0.35 mL/g, a microporous surface area of 100-300 m²/g, and a micropore volume of 0.05-0.15 mL/g. The UZSM-5 material has a smooth surface and a narrow particle size distribution, which is adjustable in the range of 200˜500 nm with a standard deviation of 10 nm.

Any two or more metals that include Ga and Mo can be used to dope the porous support material. Metals in addition to Ga and Mo that can also be provided in the porous support structure include silver (Ag), zinc (Zn), cobalt (Co), cerium (Ce), and any combinations thereof. Each metal dopant or the combination of metal dopants can be provided within the catalyst structure (e.g., in metal or metal oxide form) in an amount ranging from about 0.1 wt % to about 20 wt %. For example, Ga can be provided in the porous catalyst structure in an amount from about 0.1 wt % to about 20 wt % (e.g., from about 0.5 wt % to about 5 wt %, or from about 0.5 wt % to about 2 wt %, or about 1 wt %), while Mo (independent of Ga) can also be provided in the porous catalyst structure in an amount from about 0.1 wt % to about 20 wt % (e.g., from about 1 wt % to about 10 wt %, or from about 3 wt % to about 7 wt %, or about 5 wt %). It is noted that the term weight percentage (wt %) of metal within a catalyst structure, as described herein, refers to the mass of a particular metal element divided by the mass of the catalyst support (i.e., the mass of the porous catalyst support material before metal loading, such as the weight of a zeolite porous support material) and then multiplied by 100 (to obtain a percentage value). While other metals can be combined with Ga and Mo in a catalyst support structure, it has been determined that the combination of only the metals Ga and Mo (i.e., with no addition of any other metals) in the catalyst support structure provide a synergistic effect that will significantly enhance the desulfurization of oils such as a heavy oil, MGO or MDO in the presence of methane.

The porous support structure comprising a zeolite material can be doped with a suitable amount of two or metals (including Ga and Mo) in the following manner. Each metal salt can be dissolved in deionized water to form an aqueous solution of one or more metal precursors at a suitable concentration(s) within the solution. Metal precursor salts that can be used to form the catalyst structure include, without limitation, chlorides, nitrates, and sulfates. The metal precursors in the solution are then loaded into the porous support material to achieve a desired amount of metals within the catalyst structure (e.g., from 0.1 wt % to about 20 wt %). Any suitable loading process can be performed to load metals within the porous support material. Some non-limiting examples of metal loading processes include IWI (incipient wetness impregnation, where an active metal precursor is first dissolved in an aqueous or organic solution, the metal-containing solution is then added to catalyst support containing the same pore volume as the added solution volume, where capillary action draws the solution into the pores); WI (wet impregnation, where more liquid than the IWI volume is added to the support, and the solvent is then removed by evaporation); IE (ion-exchange, where metal cations are exchanged into the support from solution); and FI (framework incorporation, where metals are added to the support materials during the synthesis step of the support).

Depending upon the particular loading process, the resultant metal-loaded catalyst structure can be dried at a temperature between about 80° C. to about 120° C. for a period of time between about 2 hours to about 24 hours. The dried catalyst structure can then be subjected to calcination under air, N₂ or another gas or reduction under H₂ at a temperature ranging from about 300° C. to about 700° C. and at a suitable ramped or stepped increased heating rate (e.g., heating rate increases the temperature at about 5° C./min to about 20° C./min), where such calcination temperatures, times and heating rates can be modified depending upon the type or types of metals doped into the catalyst structure as well as reaction conditions associated with the use of the catalyst structure.

The catalyst structure can be processed into a granular form having a granule size as desired for a particular operation. Some examples of granular sizes include a diameter (or cross-sectional dimension) range that is about 1 mm to about 5 mm and a lengthwise or longitudinal dimension range that is about 5 mm to about 10 mm. The catalyst structure can also be formed into any other suitable configuration.

The catalyst structure can also be converted into pellets, e.g., by combining the powder into pellets using a suitable binder material. For example, the catalyst structure in powder form can be mixed with colloidal silica, methylcellulose, and a solution of an acid such as acetic acid or citric acid, where the mixture can then be extruded to form pellets. The weight ratios between catalyst powder and colloidal silica, between catalyst powder and methylcellulose, and between catalyst powder and acetic acid or citric acid solution can range from 1:0.5-2, 1:0.05-0.2, and 1:0.1-0.5, respectively. The mass concentration of acetic acid or citric acid solution can be about 10-50 wt. %. Some non-limiting examples of colloidal silica used to form the pellets include LUDOX® AM-30 and LUDOX® HS-40. Informing the pellets, the components can be added into the catalyst powder in the following order: methylcellulose, acetic or citric acid solution, and colloidal silica. In the first step, the pellet is prepared by well mixing (e.g., using a suitable mixer) of the catalyst powder and methylcellulose The acetic or citric acid solution is prepared and then combined with the catalyst mixture and the contents well mixed, followed next by the addition of colloidal silica and then further mixing. Next, the combined mixture is extruded using a suitable extruder at about room temperature (e.g., about 20° C. to about 25° C.). To control the shape and size of catalyst pellets, the extruder is equipped with a suitable forming die. In example embodiments, a catalyst pellet can have a cylindrical shape that is about 0.5 mm to about 3 mm in length and/or diameter. After extrusion, the catalyst pellet can be dried at about 80° C. to about 100° C. for about 8-12 hours, followed by calcination at 550° C. for about 12 hours (e.g., utilizing a heating rate that increases the temperature in an amount ranging from about 5-20° C./min).

The resultant metal-doped catalyst structure is suitable for use in desulfurization (and/or other upgrading processes) under a methane environment in a number of different types of batch and/or continuous processes. The catalyst structure can be utilized, e.g., for heavy oil desulfurization and/or another hydrocarbon upgrading in a number of different types of reactor systems including, without limitation, batch reactor systems, continuous tubular reactors (CTR), continuous stirred-tank reactors (CSTR), semi-batch reactors, varying catalytic reactors such as fixed bed, trickle-bed, moving bed, rotating bed, fluidized bed, slurry reactors, a non-thermal plasma reactor, and any combinations thereof.

Catalyst structures as described herein can also be regenerated, either before or after a period of time of use, to enhance the performance of the catalyst structure. The regeneration process comprises rinsing the catalyst with toluene, drying in the air to remove toluene (e.g., drying at 100° C. to about 200° C., e.g., about 150° C., for at least 1 hour, e.g., about 3 hours or greater), and calcination (heating in the air) at a temperature of at least about 500° C. (e.g., about 600° C. or greater) for a sufficient period of time, e.g., at least about 3 hours (e.g., about 5 hours or greater). The regeneration process can also be repeated any number of times depending upon a particular application. For a catalyst structure that has been used, e.g., in a desulfurization process for heavy oil, the regeneration process (e.g., single regeneration, twice regeneration, etc.) can be used to regenerate or refresh the catalyst structure such that its performance is enhanced in relation to the performance of the catalyst structure before the regeneration process. In particular, the performance of the catalytic reaction for the catalyst structure can improve when subjected to a regeneration process and after the catalyst structure has been used in long-term industrial applications. While not bound by any particular theory, it would appear that the active catalytic sites in the catalysts are further activated during the regeneration process. In particular, the metal oxides may be converted to sulfides during the reaction and better disperse in the catalyst structure. In the regeneration process, metal migration may take place to achieve a better dispersion, resulting in improved catalytic performance. The regeneration process can be repeated a plurality of times (e.g., regenerated twice, regenerated three times, etc.) for a particular application to enhance the catalytic performance of the catalyst structure.

Examples of a catalyst structure and methods for upgrading an oil (MDO and MGO) are now described.

Synthesis of a Ga—Mo UZSM-5 Catalyst Structure

A UZSM-5 catalyst with uniform cylindrical morphology was synthesized utilizing a hydrothermal technique. In particular, Al(NO₃)₃·9H₂O (98%, Alfa Aesar) was added to 1.0 M Tetrapropylammonium hydroxide (TPAOH, Sigma Aldrich) and stirred at room temperature until a clear solution was obtained. Tetraethyl orthosilicate (TEOS, Sigma Aldrich) was then added dropwise to the above solution while maintaining stirring. Upon completion of TEOS addition, the solution was left to stir for about 1 hour to allow for supersaturation. The resulting supersaturated gel was applied to a Teflon-lined autoclave and treated in a furnace at 180° C. for 72 hours. Amounts were calculated to obtain a molar ratio of Al₂O₃:80SiO₂:21TPAOH:943H₂O in the gel. After the hydrothermal synthesis, the powder was recovered by vacuum filtration and washing with deionized (DI) water 3 times. The resultant pastes were then heated at 110° C. in the oven for 8 hours and subsequently calcined in air at a rate of 5° C./min, held at 300° C. for 30 minutes, ramped at the same rate again, finally ramped at the same rate to 550° C. and held for 4 hours. The resultant UZSM-5 powder formed had a uniform particle size and compact cylindrical morphology. Then, UZSM-5 support was extruded to get the shaped pellets with a diameter of 1.0 mm and a length of 5.0 mm.

The metal modified UZSM-5 catalysts were prepared by incipient wetness impregnation of UZSM-5 support with an aqueous solution of ammonium molybdate tetrahydrate ((NH₄)₆Mo₇O₂₄·4H₂O, 99%, Alfa Aesar) and/or gallium nitrate hydrate (Ga(NO₃)₃·H₂O, Alfa Aesar), dried in the oven at 92° C. overnight, followed by calcination at 550° C. for 5 h in ambient air after each metal was loaded. The resultant catalysts were denoted as Ga/UZSM-5, Mo/UZSM-5, and Ga—Mo/UZSM-5. The amount of Ga and Mo in each catalyst structure was 1 wt % and 5 wt %, respectively.

The three different catalyst structures (Ga—Mo/UZSM-5, Ga/UZSM-5, and Mo/UZSM-5) formed in Example 1 were used for testing efficacy in desulfurization of marine gas oil (MGO) and marine diesel oil (MDO). The MGO and MDO had properties as described herein and were used as direct feedstocks (i.e., without further treatment) in the desulfurization process. Desulfurization processes can occur at operating temperatures of at least about 300° C., such as at least about 400° C., or at least about 500° C. or greater. The operating pressure for the desulfurization process can be between about 1 atm and about 200 atm.

Reactor and Reaction Conditions for Desulfurization Processes

A fixed bed reactor was used for testing desulfurization of MGO and MDO under conditions as noted herein. The reactor and reaction conditions apply to the process data as described herein and outlined in Tables 1-7.

The first feedstock used in the desulfurization processes was MGO because there are more light fractions in MGO than MDO. This feature benefits the compositional analysis using GC-MS, which cannot accurately determine the composition of very heavy fractions, to grasp a better understanding of the desulfurization process.

Four reactions with MGO as feedstock were conducted using a fixed bed reactor under a methane environment. The position of the catalyst bed was in a middle section of a reactor tube for the reactor, where the reactor tube was filled with quartz wool and glass beads at the top and the bottom section. The reactor was loaded with 5 g of catalyst pellets (formed as previously described herein) and feedstock passed the catalysts with a down-flow mode with no inert diluent. The reaction temperature was 400° C., reaction pressure was 30 bar, inlet gas flow rate was 100 sccm and feedstock pumping rate was 0.09 mL/min. Five grams (5 g) of catalyst was loaded for each reaction with a reaction time on stream of 6 hours. The catalyst employed for each run was UZSM-5, Ga/UZSM-5, Mo/UZSM-5, and Ga—Mo/UZSM-5, respectively.

Two additional reactions were conducted with the fixed bed reactor using MDO as the feedstock under a methane environment and a nitrogen environment. Both reactions used Ga—Mo/UZSM-5 as the catalyst, where all other conditions were the same as those applied in the four MGO reactions.

Characterization of Data Obtained from Processes

To obtain the mass balance of each reaction, the gas yield was calculated using the summation of the average mass of generated gas every 30 minutes divided by the mass of consumed feedstock. The necessary data to determine gas yield and methane conversion was acquired through gas chromatography (Agilent Micro-GC 490), inlet flowmeter within fixed bed reactor system, and internal standard, i.e. N₂, was added in the feed gas. The liquid yield was the ratio between the mass of the collected liquid oil and the mass of consumed feedstock.

A Thermographic Analysis (TGA) signal along with a simultaneously collected Differential Scanning calorimetry (DSC) signal combined with a simultaneous thermal analyzer (PerkinElmer STA 6000) of the spent catalyst was acquired to calculate the coke yield as well as coke formation rate.

The density of the oil samples was measured using the Anton Paar DMA 4500 M density meter. The total acid number (TAN) of the liquid sample produced from each run was measured using a Metrohm 848 Titrino Plus by averaging the results collected from at least three independent measurements.

Simulated distillation analysis of the feedstock and product oil samples was executed to compare the boiling point difference and estimate the average molecular weight, which was achieved by an Agilent 8890 GC System equipped with an analysis software SimDis Expert developed by Separation Systems. Liquid nitrogen was used to realize the cryogenic GC analysis from −20° C. to 425° C. with a ramp rate of 10° C./min. The boiling curves were calibrated using the reference sample SD-SS3E-05 supplied from Separation Systems. The average molecular weight of the feedstock and products were calculated using these simulated distillation curves.

The sulfur content was measured by a Thermo Scientific iCAP 7000 series ICP-OES spectrometer. Each sample was diluted into three different concentrations and measured at two different characteristic wavelengths to get reliable results.

Some ¹³C NMR experiments were conducted at 9.4 T (ν₀(¹³C)=100.6 MHz) on a BRUKER AVANCEIII 400 spectrometer with a BBFO 5 mm probe. ¹³C NMR chemical shifts were referenced to CDCl₃ at 77.26 ppm. A spectral width of 24 kHz and a zgig 30 pulse with a delay of 2 s were used to acquire 1024 scans per spectrum. The NMR samples in the tubes are prepared by mixing 0.1 mg sample, 0.1 mg Cr(acac)₃, and 0.5 mL CDCl₃.

Ammonia-temperature programmed desorption (NH₃-TPD) was performed to determine the surface acidity of zeolite catalysts on a chemisorption analyzer (Finesorb-3010). Typically, 0.2 g catalyst was put into a U-type quartz tube and both ends were filled with quartz wool. To remove the adsorbed impurities, temperature-programmed oxidation (TPO) test was first performed, in which the tube was heated up to 600° C. and held for 30 minutes with a ramp rate of 20° C. min⁻¹ under 5% O₂/He gas flow (flow rate 30 sccm). Then, the system was cooled down to 120° C. and ammonia adsorption was conducted by feeding 10% NH₃/He for 30 minutes (flow rate 25 sccm). Next, the physisorbed ammonia was flushed out by He gas flow for 30 minutes (flow rate 30 sccm). Finally, the desorption of ammonia was carried out from 120° C. to 800° C. with a ramping rate of 20° C. min⁻¹ and held at 800° C. for 10 minutes. The desorbed ammonia was monitored by a thermal conductivity detector (TCD) and the amount was quantified by peak integration of the corresponding calibrated TCD signal. Nitrogen adsorption-desorption analysis of catalysts was carried out on an ASAP 2020 Plus surface area and porosimeter system (Micromeritics). The sample was first degassed at 350° C. for 4 hours with a temperature ramping rate of 10° C. min⁻¹ and a vacuum level of 20 μmHg. The analysis was then performed in liquid nitrogen to get a 56-point adsorption-desorption isotherm. The total surface area was calculated by the BET method and the total pore volume was calculated at 0.995 relative pressure.

Desulfurization/Upgrading of MGO

As previously noted herein, the analysis of MGO was performed before MDO due to the greater number of light fractions present in MGO. A comparison of the performance of different catalysts all with the same support (UZSM-5), in which one catalyst included only Ga, a second catalyst included only Mo, and the third catalyst included the combination Ga—Mo, was performed using MGO with 0.2 wt % sulfur as the feedstock.

A challenge faced by the desulfurization process is the over-cracking of carbon chains while breaking the C—S bonds in the MGO molecules, resulting in a low liquid product yield. The UZSM-5 catalyst structure, having a high silica to alumina molar ratio of 80:1 and a low acid site concentration, has been determined to be effective in preserving the carbon chain structure of oil during catalytic desulfurization.

As shown in Table 1 herein, when UZSM-5 without metal loading is employed as the catalyst, 0.03% of gas yield and 97.5% of liquid yield indicate that cracking barely occurs during the reaction. In contrast, the Ga—Mo/UZSM-5 catalyst provides

TABLE 1
Mass balance results for desulfurization of MGO using
different catalyst structures

| | | | Coke | | | |
|---|---|---|---|---|---|---|
| | | | Yield | | | |
| | Gas | Liquid | in | Coke | Overall | Methane |
| | Yield | Yield | 6 h | Formation | Yield | Conversion |
| Catalyst | (wt %) | (wt %) | (wt %) | Rate (h⁻¹) | (wt %) | % |
| UZSM-5 | 0.03 | 97.5 | 0.63 | 0.006 | 98.2 | 0.03 |
| Ga/UZSM-5 | 1.62 | 95.4 | 0.93 | 0.008 | 98.0 | 0.23 |
| Mo/UZSM-5 | 3.10 | 91.7 | 1.35 | 0.012 | 96.2 | 0.27 |
| Ga- | 6.01 | 91.3 | 1.31 | 0.012 | 98.6 | 0.56 |
| Mo/UZSM-5 | | | | | | |

The data presented in Table 2 shows the degree of desulfurization (removal of sulfur) for the MGO using the different catalyst structures. Other characterization data, including TAN (total acid number), density, and average molecular weight, for the MGO product after desulfurization treatment is also provided in Table 2.

TABLE 2
Characterization results for MGO after desulfurization
using the different catalysts

| | Density | | | | Average |
|---|---|---|---|---|---|
| | at | TAN | Sulfur | | molecular |
| | 15.6° C. | (mg | content | Desulfurization | weight |
| Catalyst | (g/cm³) | KOH/g) | (ppm) | % | (g/mol) |
| None | 08524 | 0.08 | 1988 | — | 318 |
| UZSM-5 | 0.8479 | 0.08 | 1404 | 29.4 | 305 |
| Ga/ | 0.8358 | 0.07 | 1267 | 36.3 | 286 |
| UZSM-5 | | | | | |
| Mo/ | 0.8395 | 0.02 | 1156 | 41.9 | 284 |
| UZSM-5 | | | | | |
| Ga—Mo/ | 0.8430 | 0.02 | 925 | 53.5 | 275 |
| UZSM-5 | | | | | |

As is evident from the data presented in Tables 1 and 2, the Ga—Mo/UZSM-5 results in the greatest desulfurization/lowest sulfur content for the upgraded MGO product. In particular, the use of the UZSM-5 support structure results in a reduction in sulfur content from 1988 ppm to 1404 ppm (even without loading of any metal). The removal of sulfur-containing moieties is likely due to the adsorption of the sulfur atoms to the acidic sites in the zeolite framework, as is evidenced by the trivial coke yield. Methane conversion with the UZSM-5 (no metal loading) catalyst is very small (almost 0) due to the fact that CH₄ cannot be activated without the presents of active metal sites for the catalyst structure.

The presence of Ga in the modified UZSM-5 (Ga/UZSM-5) catalyst structure enhances the desulfurization activity of the catalyst by lowering the sulfur content in the product to 1267 ppm (Table 2), while the liquid product yield remains at a high level of 95.4% (Table 1). The presence of Mo in the modified UZSM-5 (Mo/UZSM-5) catalyst structure can provide anchor sites for sulfur atoms, where the sulfur content of the product is reduced to 1156 ppm (Table 2). In addition, Mo loaded catalyst structure yields an upgraded MGO product in which the total acid number (TAN) is decreased to 0.02 mg KOH/g from 0.08 mg KOH/g of the feedstock, equivalent to a 75% reduction of the acid groups in feedstock molecules, which may be closely related to the conversion of sulfur-containing groups. The liquid product yield is 91.7%, slightly lower than that from Ga/UZSM-5, indicating that the cracking of diesel molecules becomes more significant.

The combination of Ga—Mo in the catalyst structure (Ga—Mo/UZSM-5) in the desulfurization process for treating MGO results in much more significant sulfur removal from the combination of these two metals. In particular, the sulfur content is decreased to as low as 925 ppm (Table 2), indicating a synergistic effect of the Ga and Mo components in the desulfurization process, particularly when used in the UZSM-5 structure. Along with the sulfur content, the density of the products is also reduced after the reactions. Methane activation and desulfurization appear to happen simultaneously on the surface of the catalyst. With this Ga—Mo combined catalyst structure, it is believed that the sulfur species are adsorbed on the Mo sites while the adjacent Ga sites catalyze the CH₄ molecules to form H and CH₄₋ₓ moieties. Those species can help to form H₂S with those adsorbed sulfur species and released into the gas phase leading to the creation of sulfur vacancies surrounding Mo atoms. Therefore, the overall desulfurization ability is greatly enhanced by using this catalyst structure with the Ga—Mo metal combination.

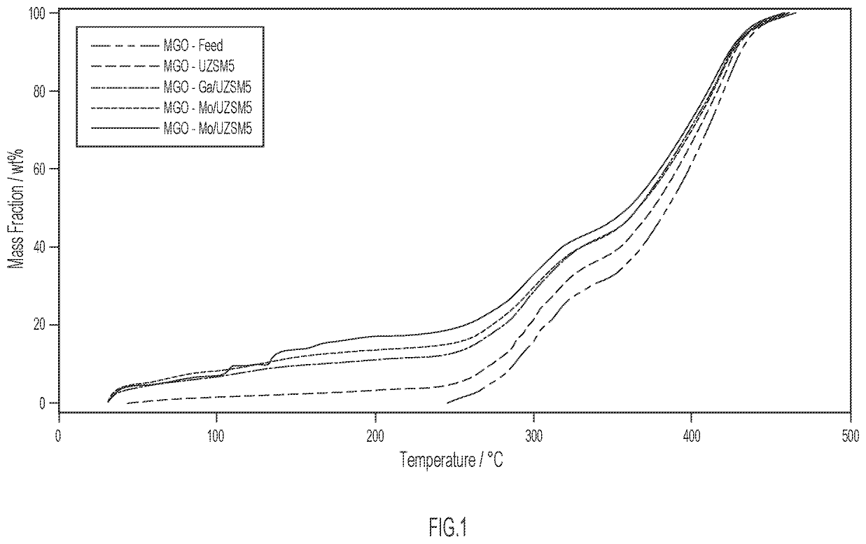

FIG. 1 presents simulated distillation curves of the MGO feedstock and the products formed by the desulfurization process using the different catalyst structures and based upon the average molecular weight (AMW) data for the products as outlined in Table 2. It was observed that the feedstock has the highest boiling point distribution and the highest AMW of 318.3 g/mol. After the reaction over UZSM-5, the distillation curve slightly moves toward the low-temperature region (i.e., more diesel fractions are distilled at a given temperature), indicating a lowered boiling point of the product matrix. The AMW is also reduced to 304.7 g/mol, indicating cracking and the removal of sulfur atoms from the product molecules during the reaction. When Ga/UZSM-5 and Mo/UZSM-5 are used to catalyze the reaction, the distillation curves further move to lower temperature regions and AMW values are reduced to 286.3 g/mol and 283.8 g/mol, respectively. When Ga—Mo/UZSM-5 is employed as the catalyst, the distillation curve is above those obtained from other conditions and the AMW is only 275.1 g/mol, indicating that the product molecules become lighter upon the desulfurization process over Ga—Mo/UZSM-5.

The density of the MGO feedstock is 0.8524 g/cm³ at 15.6° C., which is reduced to 0.8358 g/cm³, 0.8395 g/cm³, and 0.8430 g/cm³ after the reaction over Ga/UZSM-5, Mo/UZSM-5, and Ga—Mo/UZSM-5, respectively. The sulfur-containing groups increase the polarity of the molecules, which is related to the dipole-dipole force and the induction force between the molecules. As the sulfur-containing groups are converted, the Van der Waals interaction between the product molecules via these groups may be suppressed. As a consequence, the density of the product is reduced after the reaction.

When comparing the density values of product oil after the reaction using Ga/UZSM-5, Mo/UZSM-5, and Ga—Mo/UZSM-5 as the catalyst, it is interesting to find that the density values follow the order of Ga/UZSM-5<Mo/UZSM-5<Ga—Mo/UZSM-5. However, the product from Ga—Mo/UZSM-5 demonstrates the lowest sulfur content and boiling point, while that from Ga/UZSM-5 has the highest sulfur content and boiling point. To better understand this phenomenon, a more thorough analysis of the products was conducted using gas chromatography-mass spectrometry (GC-MS) analysis. Because of the high boiling points of the heavy fractions in the product, only compounds with boiling points below 250° C. could be accurately quantified, while heavier compounds could not be separated by the GC column. However, an indication of the aromatization process taking place during the reaction can be achieved by determining the selectivity of aromatic product molecules including benzene, toluene, ethylbenzene, and xylenes (BTEX). Table 3 provides BTEX characterization data of the upgraded MGO products using the different catalyst structures.

TABLE 3
Aromatics selectivity of the MGO products subjected to
desulfurization (distillates < 250° C.)

| | Benzene | Toluene | Ethylbenzene | Xylene | Aromatics |
|---|---|---|---|---|---|
| Catalyst | (wt %) | (wt %) | (wt %) | (wt %) | (wt %) |
| UZSM-5 | 0 | 0 | 0 | 0 | 0 |
| Ga/UZSM-5 | 0 | 6.2 | 0 | 8.0 | 14.2 |
| Mo/UZSM-5 | 0 | 11.1 | 3.9 | 18.3 | 50.63 |
| Ga—Mo/ | 2.7 | 16.5 | 4.8 | 25.6 | 75.2 |
| UZSM-5 | | | | | |

After the reaction over Ga—Mo/UZSM-5, the selectivity of aromatics is as high as 75.2% among the product molecules with boiling points below 250° C. The selectivity of BTEX is as high as 49.6%, which makes the upgrading more profitable since BTEX is a valuable feedstock in petrochemical production. When Ga/UZSM-5 and Mo/UZSM-5 are used as the catalyst, the aromatic product selectivity is 14.2% and 50.6%, respectively. On the other hand, a negligible amount of BTEX is observed in the feedstock and the product obtained from the reaction using UZSM-5 as the catalyst.

In addition to data for the light distillates, carbon-13 nuclear magnetic resonance (¹³C NMR) spectra of the feedstock and product samples were acquired to evaluate the aromatization of all the fractions in the sample. Table 4 provides the BTEX spectral data the (¹³C peak area assigned to carbon atoms in phenyl rings and that due to paraffin and substitution groups) for the MGO products upgraded with the different catalyst structures.

TABLE 4
¹³C NMR peak area percentages of MGO and the products assigned
to carbons in phenyl rings and paraffin the substitution groups

| | Phenyl ring | Paraffin and substitution group |
|---|---|---|
| Catalyst | (%) | (%) |
| None | 0 | 100 |
| UZSM-5 | 0 | 100 |
| Ga/UZSM-5 | 8.6 | 91.4 |
| Mo/UZSM-5 | 8.9 | 91.1 |
| Ga—Mo/UZSM-5 | 13.2 | 86.8 |

From the spectral data, it can be seen that 13.2% of the carbon atoms in the MGO product obtained using the Ga—Mo/UZSM-5 catalyst structure are attributed to phenyl rings, while only 8.6% and 8.9% carbon atoms are in phenyl rings when Ga/UZSM-5 and Mo/UZSM-5 are employed as the catalyst structures to upgrade the MGO feedstock. A significantly improved aromatization is indicated with Ga—Mo/UZSM-5 as the catalyst, which is in line with the compositional analysis results from GC-MS analysis. These results indicate that the aromatization process is more significant when Ga—Mo/UZSM-5 is the catalyst. The π-interaction between the phenyl rings enhances the interaction between the aromatic molecules, resulting in increased density of the product matrix. Therefore, the highest density of product obtained over Ga—Mo/UZSM-5 is observed along with the lowest distillation temperature and sulfur content.

Thus, the data provided show the highly effective desulfurization activity of the UZSM-5 catalyst structure that utilizes a combination of Ga and Mo. The cracking of the MGO molecules is not severe with liquid product yields above 90% after the reactions. As desulfurization takes place, the conversion of sulfur-containing groups results in the reduction of TAN as well as the dipolar interaction. The MGO molecules are converted to smaller molecules with fewer sulfur-containing groups as the reaction proceeds when the Ga—Mo UZSM-5 catalyst structure is used. Further, the sulfur content, boiling point, average molecular weight, and density of the product are decreased as a consequence. In addition to the reduction of sulfur content, the aromatization process of the light fraction molecules leads to a considerable amount of BTEX products utilizing the Ga—Mo UZSM-5 catalyst structure.

Desulfurization/Upgrading of MDO

The significantly enhanced desulfurization and other upgrading results achieved with MGO were further tested using MDO as the feedstock, where Ga—Mo/UZSM-5 catalyst was used in the desulfurization process with methane and with nitrogen substituted for methane. The MDO feedstock comprised a blended fuel oil consisting of MGO as the major component and a very small portion of heavy fuel oil (HFO), where the sulfur content of the MDO feedstock was 2153 ppm. The mass balance results and characterization results are provided in Table 5 and Table 6, respectively.

TABLE 5
Mass balance results for desulfurization of MDO using
Ga-Mo/UZSM-5 catalyst in methane and nitrogen environments

| | | | | Coke | | | |
|---|---|---|---|---|---|---|---|
| | | | | Yield | | | |
| | | Gas | Liquid | in | Coke | Overall | Methane |
| | Feed | Yield | Yield | 6 h | Formation | Yield | Conversion |
| Catalyst | gas | (wt %) | (wt %) | (wt %) | Rate (h⁻¹) | (wt %) | % |
| Ga- | CH₄ | 2.56 | 96.1 | 1.20 | 0.011 | 99.86 | 1.07 |
| Mo/UZSM-5 | | | | | | | |
| Ga- | N₂ | 2.82 | 95.4 | 1.34 | 0.012 | 99.57 | — |
| Mo/UZSM-5 | | | | | | | |

TABLE 6
Characterization results for MDO after desulfurization using
Ga—Mo/UZSM-5 catalyst in methane and nitrogen environments

| | Density | | | | Average |
|---|---|---|---|---|---|
| | at | TAN | Sulfur | | molecular |
| | 15.6° C. | (mg | content | Desulfurization | weight |
| Sample | (g/cm³) | KOH/g) | (ppm) | % | (g/mol) |
| MDO | 0.85239 | 0.24 | 2153 | 0 | 375 |
| Feedstock | | | | | |
| MDO | 0.84176 | 0.03 | 887 | 58.8 | 281 |
| product | | | | | |
| (CH₄ | | | | | |
| environment) | | | | | |
| MDO | 0.83456 | 0.05 | 1231 | 42.8 | 269 |
| product | | | | | |
| (N₂ | | | | | |
| environment) | | | | | |

The data provided in Tables 5 and 6 indicate that, when Ga—Mo/UZSM-5 is employed as the catalyst in methane, the sulfur content of the MDO product from the desulfurization process is reduced to 887 ppm, equivalent to a 58.8% sulfur content reduction, which is even greater compared with the desulfurization performance using MGO as the feedstock. A greater methane conversion (when using methane environment for the desulfurization process) results in a greater methane conversion of 1.8% compared for the MDO product compared with the 0.4% methane conversion when MGO is the feedstock. The TAN is decreased to 0.03 mg KOH/g from 0.24 mg KOH/g of the MGO feedstock. In addition, the liquid yield of the MDO reaction is higher than the MGO reaction under the same conditions, indicating that more methane might be activated and incorporated into the products. Both the gas and coke yields are lowered compared with those derived from the MGO counterpart due to the suppression of over-cracking by the participation of more prominent methane engagement, leading to a higher liquid yield of 96.1% (Table 5). These phenomena indicate that the catalytic desulfurization under methane is even more enhanced using MDO as the feedstock.

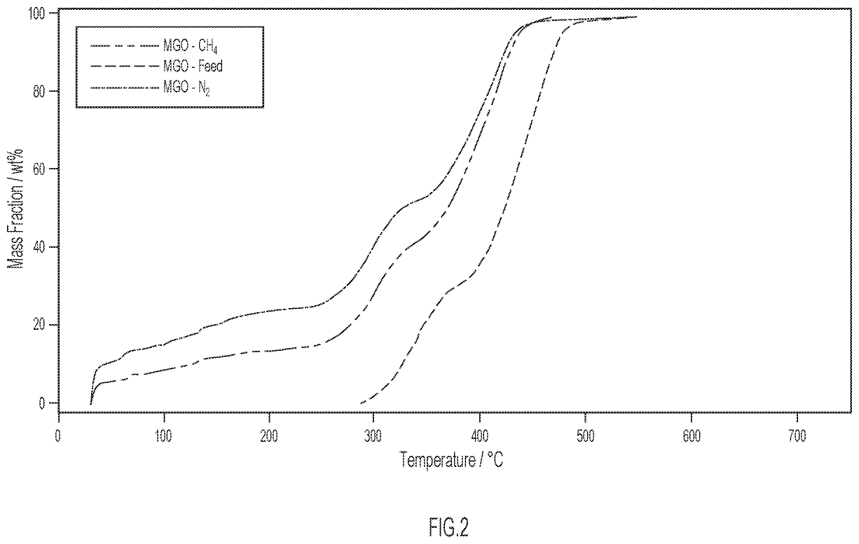

To verify the contribution of the activated methane molecules in the desulfurization process, the control experiment with Ga—Mo/UZSM-5 catalyst under N₂ was carried out. The sulfur content under the nitrogen environment is 1231 ppm in the product oil, much higher than that obtained under the CH₄ environment of 887 ppm (Table 6). The liquid product yield is 95.4%, lower than that obtained under the CH₄ environment of 96.1% (Table 5), while the gas yield is higher than the CH₄ environment counterpart. These phenomena show that the participation of methane in the desulfurization process not only enhances the removal of sulfur species but also improves liquid product selectivity. By comparing the simulated distillation curves of the MDO feedstock and the product oil samples obtained under N₂ and CH₄ environment, which are shown in FIG. 2, it is clear that the boiling point is decreased after the upgrading/desulfurization process. The average molecular weight is decreased from 375 to 268 and 281 g/mol, respectively (Table 6). It is clear that the distillation temperature, average molecular weight, and density of the product obtained under the methane environment are higher, indicating incorporation of methane into the liquid product molecules and the suppressed over-cracking of MDO molecules under the methane environment. These phenomena demonstrate the significance of methane participation in the desulfurization process for MDO in terms of improved conversion of sulfur atoms and increased liquid product yield.

Characterization of UZSM-5 Catalyst Structure

The porosity properties of UZSM-5 and Ga—Mo/UZSM-5 were compared, and the data is provided in Table 7 as follows.

TABLE 7
Porosity properties of UZSM-5 and Ga—Mo/UZSM-5 catalysts

| | BET surface area | Total pore volume |
|---|---|---|
| Catalyst | (m²/g) | (ml/g) |
| UZSM-5 | 321 | 0.258 |
| Ga—Mo/UZSM-5 | 287 | 0.224 |
| Ga—Mo/UZSM-5 (spent) | 224 | 0.105 |

The shaped UZSM-5 support has a typical surface area and pore volume for an MFI type zeolite structure. As indicated in Table 7, the loading of Ga and Mo results in slightly lower surface area and pore volume for the structure. A spent (used) catalyst has a clear decrease in both the surface area and pore volume due to the formation of coke during the reaction, which is about 1.2% after 6 h for the desulfurization process of MDO (Table 5). The decrease of the surface area is about 22%, while the decrease of the pore volume is about 53%, implying that the majority of coke species are present at the mesoporous interstices between the zeolite particles.

An NH₃-TPD study of UZSM-5 and Ga—Mo/UZSM-5 catalyst structures was also conducted to better understand the effect of metal loading on the catalysts in terms of acidity. The NH₃ desorption peaks of both samples appear below 250° C., indicating that the acidic sites in these samples demonstrate weak acidity. After quantification, the total acid amounts for UZSM-5 and Ga—Mo/UZSM-5 catalysts were determined to be 114 and 527 μmol NH₃/g_{cat}, respectively. The modification by Ga and Mo to the UZSM-5 structure significantly increased the number of acid sites, which indicates the enhancement in catalytic activity for desulfurization reactions during the desulfurization process of oil, such as heavy oil, MGO or MDO.

Thus, the use of a catalyst structure as described herein that includes a combination of Ga and Mo provides a significantly enhanced reduction of sulfur from a sulfur-containing feedstock, in particular an oil feedstock such as MGO or MDO, as well as other effective upgrading features (liquid yield, TAN, density, etc.) to the heavy oil production. The use of a methane environment also enhances the desulfurization process, as does provide a porous aluminosilicate support structure for the catalyst metals as described herein. Over 50% sulfur reduction for MGO and MDO can be achieved when using a Ga—Mo/UZSM-5 catalyst under a methane environment.

While the invention has been described in detail and with reference to specific embodiments thereof, it will be apparent to one skilled in the art that various changes and modifications can be made therein without departing from the spirit and scope thereof.

Thus, it is intended that the present invention covers the modifications and variations of this invention provided they come within the scope of the appended claims and their equivalents.

What is claimed:

1. A method of desulfurizing a sulfur-containing hydrocarbon feedstock, the method comprising introducing the sulfur-containing hydrocarbon feedstock within a reactor in the presence of a gas atmosphere and a catalyst structure, wherein the catalyst structure comprises a zeolite porous support structure including gallium (Ga) and molybdenum (Mo) loaded in the zeolite porous support structure.

2. The method of claim 1, wherein the gas atmosphere comprises methane.

3. The method of claim 1, wherein the sulfur-containing hydrocarbon feedstock comprises a heavy oil.

4. The method of claim 1, wherein the sulfur-containing hydrocarbon feedstock comprises a marine gas oil or marine diesel oil.

5. The method of claim 1, wherein each of Ga and Mo is loaded in the zeolite porous support structure in an amount from about 0.1 wt % to about 20 wt % based upon the weight of the zeolite porous support material.

6. The method of claim 1, wherein the zeolite porous support structure comprises a ZSM-5 material.

7. The method of claim 6, wherein Ga is provided in an amount from about 0.5 wt % to about 2 wt % by weight of the ZSM-5 material, and Mo is provided in an amount from about 3 wt % to about 7 wt % by weight of the ZSM-5 material.

8. The method of claim 6, wherein Ga is provided in an amount of about 1 wt % by weight of the ZSM-5 material, and Mo is provided in an amount of about 5 wt % by weight of the ZSM-5 material.

9. The method of claim 6, wherein metals loaded within the ZSM-5 material consist of Ga and Mo.

10. The catalyst structure of claim 1, wherein the zeolite porous support structure has a ratio of silicon oxide to aluminum oxide from 1:1 to 280:1.

11. The catalyst structure of claim 1, wherein the zeolite porous support structure has a ratio of silicon oxide to an aluminum oxide of 80:1.

12. The method of claim 1, wherein the desulfurizing of the sulfur-containing hydrocarbon feedstock is performed in a batch reactor or a continuous reactor.

13. The method of claim 1, wherein the desulfurizing of the sulfur-containing hydrocarbon feedstock is performed in a fixed bed reactor at a temperature of at least about 400° C.

14. The method of claim 13, wherein the desulfurizing of the sulfur-containing hydrocarbon feedstock is further performed at a pressure within the fixed bed reactor that is between about 1 atm and about 200 atm.

15. The method of claim 1, wherein the catalyst structure comprises a plurality of pellets.

16. The method of claim 1, wherein the desulfurizing of the sulfur-containing hydrocarbon feedstock results in a reduction of at least about 50% by weight of sulfur content from the feedstock.

17. The method of claim 15, wherein the desulfurizing of the sulfur-containing hydrocarbon feedstock further results in a change in one or more of the following in the feedstock: density, viscosity, total acid number, one or more aromatic hydrocarbons, hydrogen to carbon ratio, and cetane number.
